(19) United States
(12) Patent Application Publication
(10) Pub. No.: US 2006/0079490 A1
(43) Pub. Date: Apr. 13, 2006

(54) Method of treatment of type I diabetes

(76) Inventors: Hector F. DeLuca, Deerfield, WI (US); Ehren N. Rudolph, Madison, WI (US); Laura McCary Bloss, Oxnard, CA (US); Julia B. Zella, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/231,049

(22) Filed: Sep. 20, 2005

Related U.S. Application Data
(63) Continuation-in-part of application No. 09/769,579, filed on Jan. 25, 2001.

(51) Int. Cl. A61K 31/59

(52) U.S. Cl. 514/167

(57) ABSTRACT

A method of delaying the onset or reducing the severity of diabetes in a human patient is disclosed. In one embodiment, the invention comprises the step of orally administering to the human patient an effective amount of a vitamin D compound such as the onset of diabetes or diabetes symptoms is slowed.

### CROSS-REFERENCE TO RELATED APPLICATION

This application is a continuation-in-part of U.S. Ser. No. 09/769,579, filed Jan. 25, 2001, incorporated by reference as if fully set forth herein.

### STATEMENT REGARDING FEDERALLY SPONSORED RESEARCH OR DEVELOPMENT

### BACKGROUND OF THE INVENTION

Diabetes Mellitus

Diabetes mellitus is a heterogenous disease that is typically characterized on the basis of a patient's hypoglycemia. In the late 1960s, a distinction was made between insulin-dependent diabetes mellitus (Type I) and non-insulin-dependent diabetes mellitus (Type II).

Type I diabetes is known to have an autoimmune origin and be influenced by both genetic predisposition environmental factors (J. F. Bach, Endoc. Rev. 18(4):516-542, 1994). Type I diabetes is a hereditary disease with a relatively high rate of familial transmission.

Environmental factors seem to influence Type I diabetes onset and may alter the course of the disease. For example, more that 60% of identical twins differ in their susceptibility to the disease. Additionally, the disease frequency varies enormously from the country to country and some non-immunological interventions seem to increase or decrease the disease rate in animal models. These interventions include specific diets and several viral infections.

Animal Models of Type I Diabetes

The non-obese diabetic (NOD) mouse is used as a model of human Type I diabetes because destruction of the islet cells occurs via an autoimmune reaction in both. A characteristic of this diabetes is termed “insulitis,” the infiltration of lymphocytes into the pancreas, indicating an immune response.

Development of Type I diabetes in the NOD mouse is T-cell mediated, involving the participation of both CD8+ and CD4+ cells (L. S. Wicker, et al., Annu. Rev. Immunol. 13:179-200, 1995). Two critical islet cell autoantigens are glutamic acid decarboxylase (GAD) and insulin. GAD catalyzes the production of the neurotransmitter, gamma-aminobutyric acid, and antibodies to GAD are often found in the sera of pre-diabetics (S. Baekkeskov, et al., Nature 347:151-156, 1990; W. A. Hagopian, et al., J. Clin. Invest. 91:368-374, 1993).

Autoantibodies to insulin also play a critical role in the onset of diabetes. These antibodies are found in about 50% of recent-onset diabetics (L. Castano and G. S. Eisenbarth, Annu. Rev. Immunol. 8:647-680, 1990). Characteristics of Type I diabetes include hyperglycemia, increased thirst and urine production, increased cholesterol in the blood, and increased blood triglyceride concentration. Type I diabetes is not usually associated with obesity.

Despite the 100% genetic similarity in the NOD mouse, only 70-80% of chow-fed NOD females develop diabetes and only 20% of chow-fed males develop the disease (S. Makino, et al., Exp. Anim. 30:137-140, 1981). Similarly, in humans, in only 50% of the cases do both twins develop diabetes (A. H. Barnett, et al., Diabetologia 20:404-409, 1981). Therefore, there must be both genetic and environmental contributions to the development of Type I diabetes. It is known that in the NOD mouse, a gene linked to the major histocompatibility complex (MHC) is involved in diabetes development, but this gene is not sufficient to cause disease; thus, more than one gene is involved in the development of diabetes incidence in the NOD mouse (L. S. Wicker, et al., supra, 1995) (H. Acha-Orbea and H. O. McDevitt, Proc. Natl. Acad. Sci. USA 84:2435-2439, 1987; J. Todd, et al., Nature 329:599-604, 1987). Specifically, as many as 19 genetic regions may be involved in susceptibility to diabetes as determined by linkage studies (T. J. Vyse and J. A. Todd, Cell, 1996). Some of these same regions have been identified as being involved in two other autoimmune diseases, systemic lupus erythematosus and experimental autoimmune encephalomyelitis (T. J. Vyse and J. A. Todd, supra, 1996). Environmentally, a north-south gradient exists in the development of diabetes with the highest incidence being in northern Europe and decreased incidence in more southern or tropical locations (A. S. Krolewski, Diabetes 37(8):1113-1119, 1988; A. S. Krolewski, et al., New Eng. J. Med. 317:1390-1398, 1987).

### SUMMARY OF THE INVENTION

In one embodiment, the present invention is a method of delaying the onset of diabetes in a human patient, comprising the step of orally administering to the patient an effective amount of a vitamin D compound such that the onset of Type I diabetes or diabetes symptoms is slowed or eliminated. We have determined that there is a preferable window of treatment for diabetes I patients. The patient would preferably be treated after the development of at least two autoantibodies associated with diabetes, most notably selected from autoantibodies to insulin, glutamic acid decarboxylase, or insulinoma-associated protein. Preferably, the treatment should begin before the patient has developed a blood glucose level of 150 mg/dl. Preferably, the amount of time from development of diabetes-related antibodies should not exceed one year.

In a preferred embodiment, the vitamin D compound is selected from the group consisting of 1α,25-dihydroxyvitamin D₃ (1,25-(OH)₂D₃), 19-nor-1,25-dihydroxyvitamin D₂ (19-nor-1,25-(OH)₂D₃), 24-homo-22-dehydro-22E-1α,25-dihydroxyvitamin D₃ (24-homo-22-dehydro-22E-1,25-(OH)₂D₃), 1,25-dihydroxy-24(E)-dehydro-24-homo-vitamin D₃ (1,25-(OH)₂-24-homo D₃), 19-nor-1,25-dihydroxy-21-epi-vitamin D₃ (19-nor-1,25-(OH)₂-21-epi-D₃), la hydroxy vitamin D₃ or 1α hydroxy vitamin D₂.

In another preferred embodiment, we envision that the following compounds would be useful: 1α,25-dihydroxyvitamin D₃, 1α hydroxyvitamin D₃, 1α,25-dihydroxyvitamin D₂, 1α-hydroxyvitamin D₂, 19-nor-1α-hydroxyvitamin D₂, 22-oxa-1α,25 dihydroxyvitamin D₃, 26,27-hexaflouro-1α,25 dihydroxyvitamin D₃, 24R-hydroxy-26,27-cyclo-1α hydroxyvitamin D₃, 2-methylene-19-nor-(20S)-1α,25 dihydroxyvitamin D₃, 2 methylene-19 nor-20S-1α,25 cihydroxy vitamin D₃ (2MD), 2α methyl-19 nor-20S-1α,25 hydroxy vitamin D₃, and their 2R isomers, 2 methylene 19 nor, 1α hydroxyl, 2 methylene-19 nor-1α hydroxy homo pregnacalciferol, and 2 methylene-19 nor-1α hydroxyl bis homo pregnacalciferol.

In another preferred embodiment, the oral administration is via diet and between 0.0016-0.833 μg, preferably between 0.005 μg-0.2 μg per kilogram of patient weight per day.

In another embodiment the present invention is a method of reducing the severity of diabetes symptoms comprising orally administering to a human diabetes patient an effective amount of vitamin D compounds such that diabetes symptoms are lessened.

### DETAILED DESCRIPTION OF THE SEVERAL VIEWS OF THE DRAWINGS

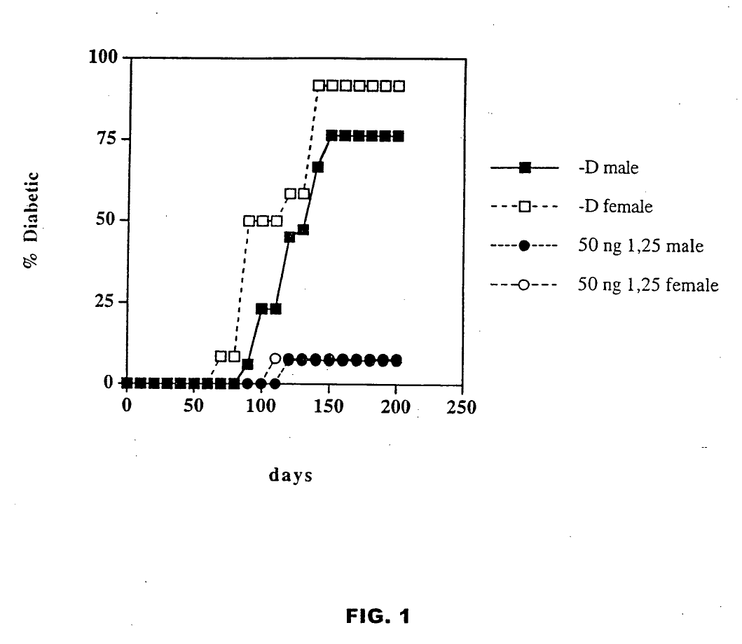

FIG. 1 graphs the incidence of diabetes as calculated as the percentage of animals demonstrating serum glucose measurements above 300 mg/dL in weekly bleeds of the NOD mice. Animals were first bled at 40 days of age, and then weekly thereafter.

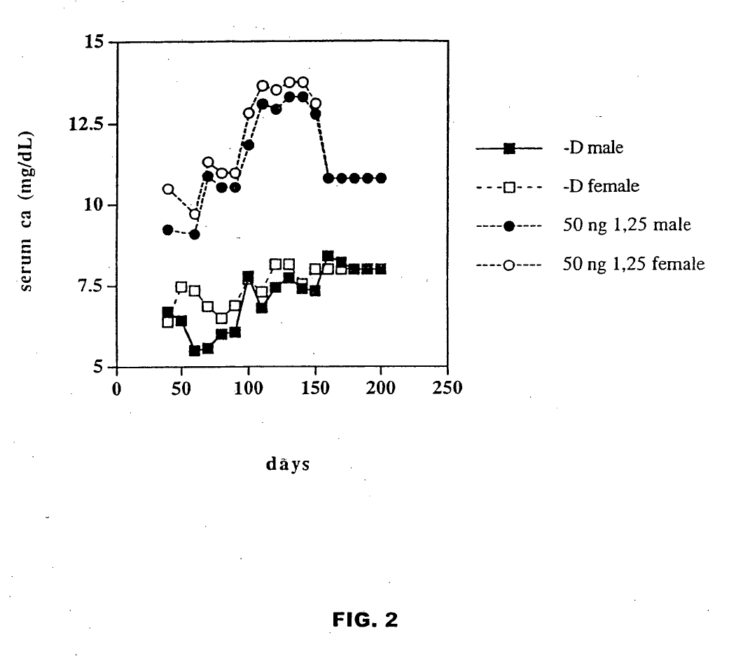

FIG. 2 graphs the results of serum calcium measurements performed weekly in the NOD mice of FIG. 1 beginning at 40 days of age. Data are expressed as mg/dL of serum calcium.

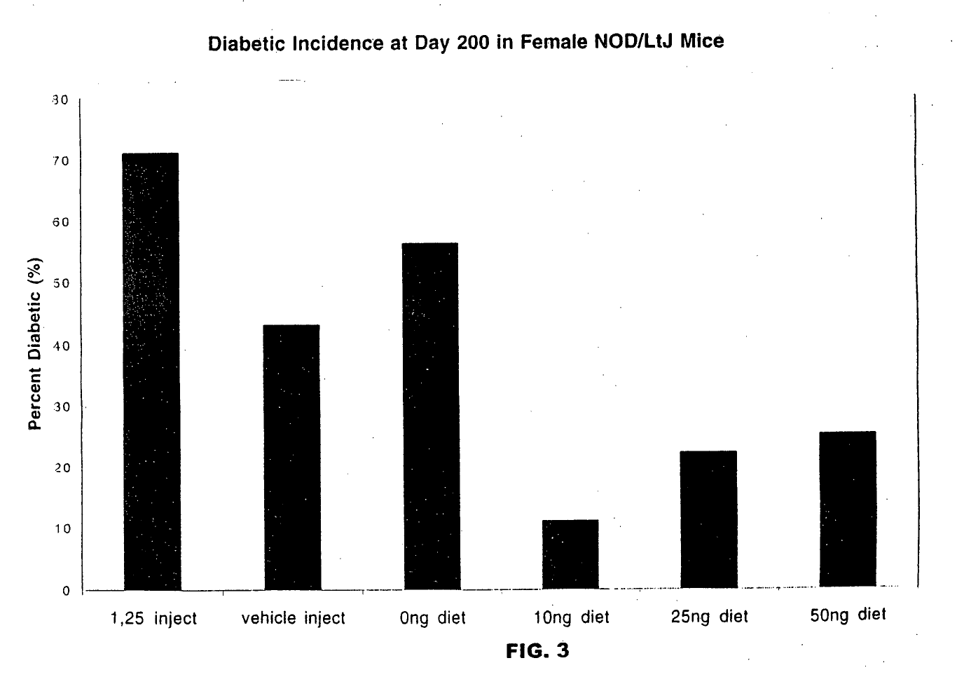

FIG. 3 is a bar graph describing diabetic incidence at day 200 in females NOD/LTJ mice.

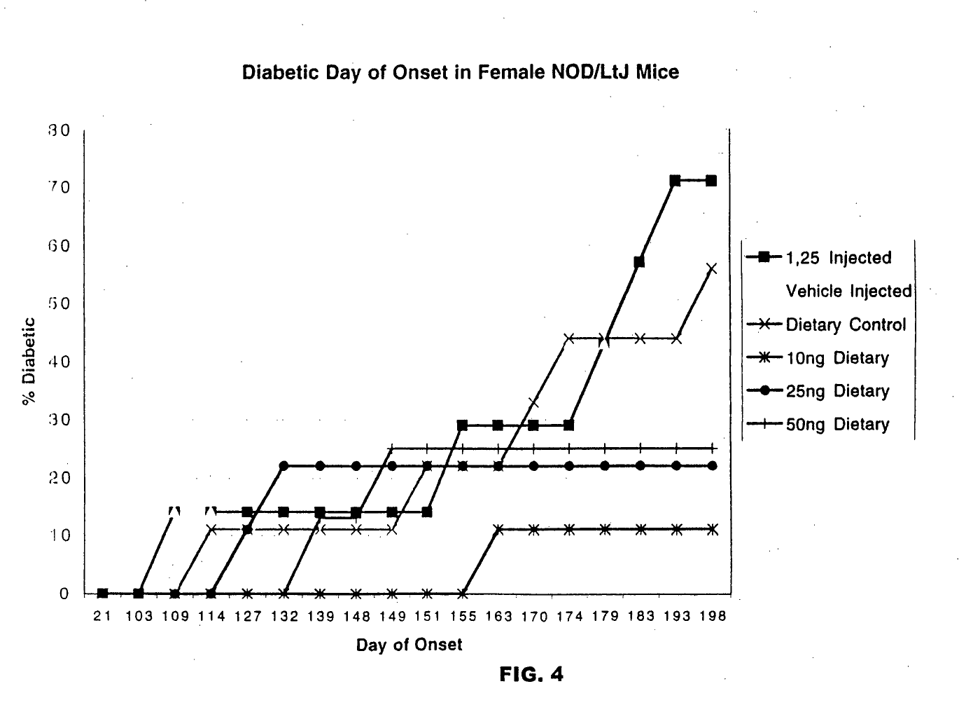

FIG. 4 is a graph demonstrating diabetic day of onset in female NOD/LTJ mice.

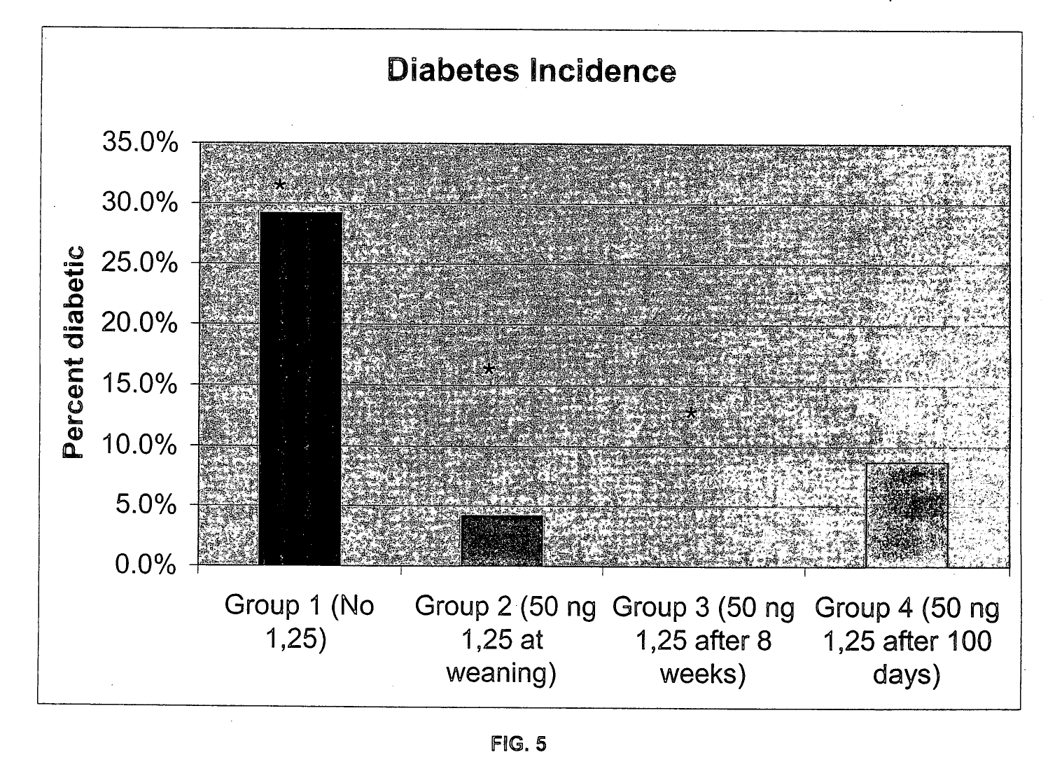

FIG. 5 is a bar graph describing diabetes incidence among four groups of NOD rats. The groups differ in the amount of 1,25(OH)₂D₃ that has been administered and the timepoint at which treatment begins.

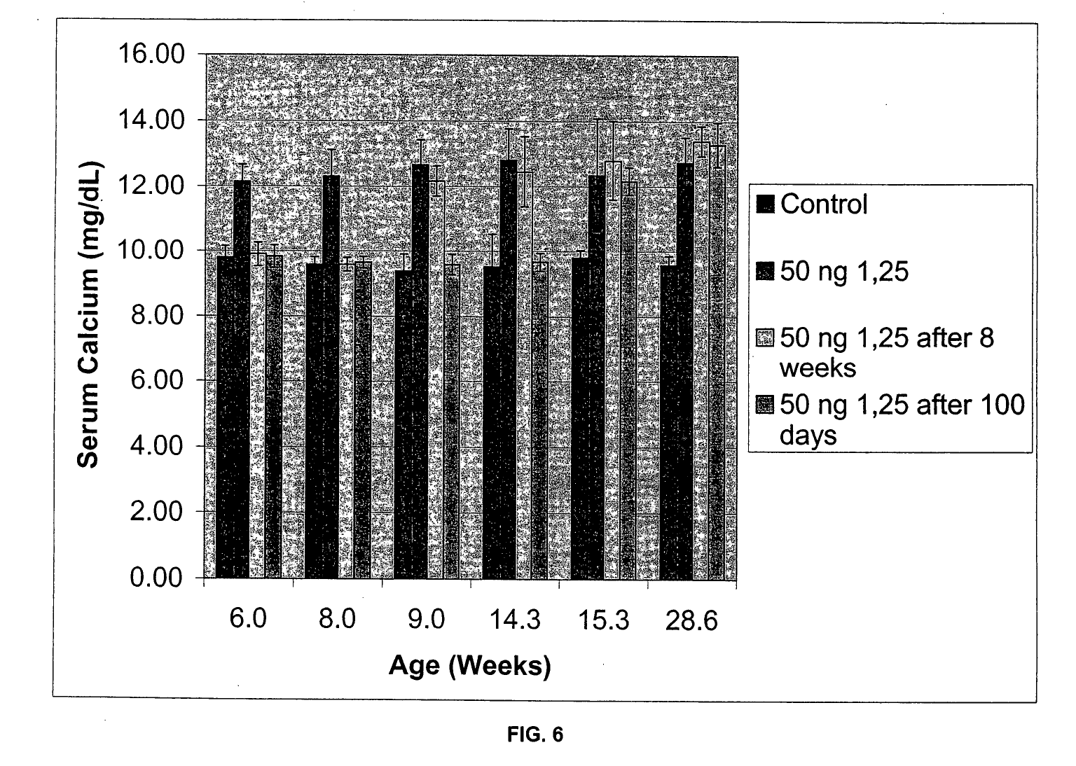

FIG. 6 is a bar graph showing serum calcium levels in the four groups of FIG. 5.

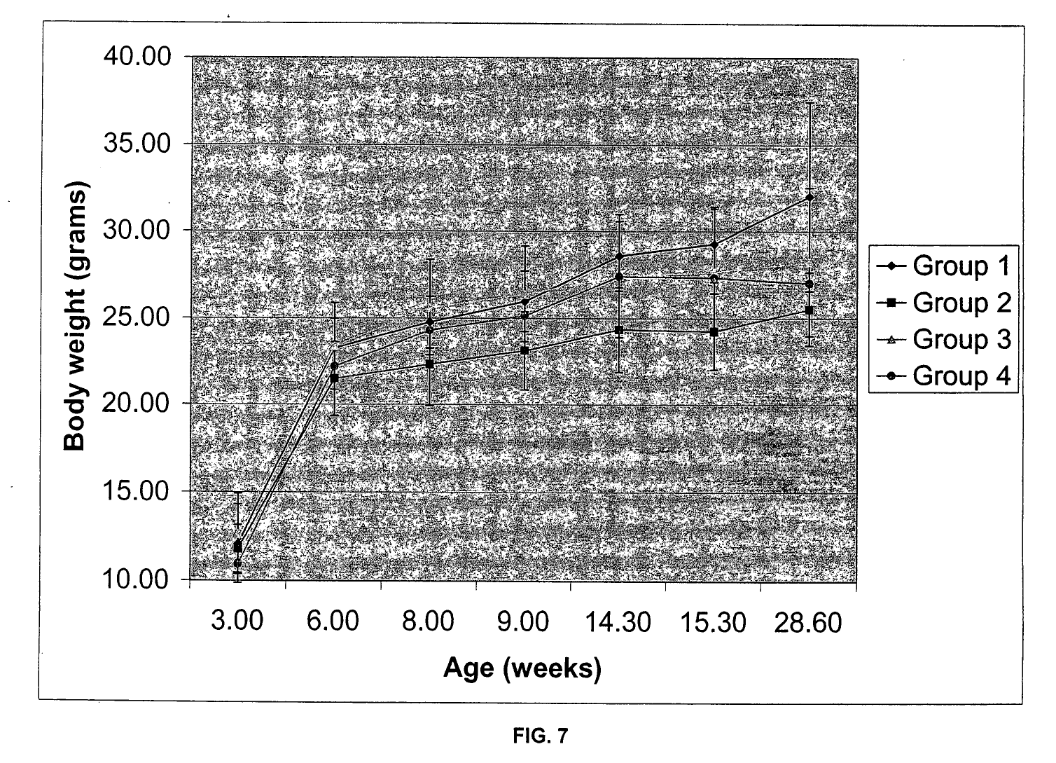

FIG. 7 graphs body weight as a function of age in the four groups of FIG. 5.

### DETAILED DESCRIPTION OF THE INVENTION

In a prior publication, Mathieu, et al. asserted that injection of 5 μg/kg 1,25(OH)₂D₃ reduced incidence of diabetes from 56% to 8% in female NOD mice (C. Mathieu, et al., Diabetologia 37:552-558, 1994). The authors proposed that 1,25(OH)₂D₃ was correcting a defective immune suppressor function by reducing T-lymphocyte proliferation and interleukin-2, interferon γ, and tumor necrosis factor a secretion (C. Mathieu, et al., supra, 1994). Mathieu, et al. treated NOD mice with 1,25(OH)₂D₃ only until 100 days of age and found that total diabetes incidence in these mice was about 35% at 200 days as compared to 86% in control mice and 13% in mice treated with 1,25(OH)₂D₃ until 200 days of age. Administering 1,25(OH)₂D₃ from 100 to 200 days of age resulted in the same diabetic incidence as the control group but the onset of diabetes appeared to be slightly delayed. Bone calcium was reduced in the 1,25(OH)₂D₃-treated mice, but these animals were given a low-calcium diet (0.2%) to prevent hypercalcemia (C. Mathieu, et al., “Vitamin D and Diabetes.” In: Vitamin D, D. Feldman, F. H. Glorieux, and J. W. Pike, eds. Academic Press, San Diego, Chapter 70, pp. 1183-1196, 1997).

In contrast, we have found a dramatically decreased incidence of diabetes in both male and female NOD mice treated with oral 1,25(OH)₂D₃ as opposed to vitamin D-deficient controls. Surprisingly, in the vitamin D-deficient NOD mouse colony, the incidence of diabetes was increased as compared to published incidence in the NOD mice. This indicates that in vitamin D-deficiency, there is more of a genetic contribution to diabetes incidence and not as much of an environmental contribution. The animals receiving 1,25(OH)₂D₃ did develop hypercalcemia. A preferable treatment would be an analog of 1,25(OH)₂D₃ that is immunoreactive, but not as calcemic.

In a preferred version of the present invention, the vitamin D compound is administered after the development and detection of autoantibodies to at least two diabetes antigens, preferably those selected from the group consisting of insulin, glutamic acid decarboxylase, and insulinoma associated protein 2. Most preferably, the administration of the vitamin D compound begins before the patient develops the high blood sugar that characterizes diabetes. By “high blood sugar,” we mean a blood sugar measurement of an average of at least 150 mg/dl blood glucose taken from five samples spaced throughout the day at least 2 hours apart. Most preferably, the treatment begins within 1 year of occurrence of these autoantibodies.

Therefore, in one embodiment, the present invention is a method of treating human Type I diabetes patients by orally administering an amount of vitamin D compound, preferably 1,25(OH)₂D₃ or analogs thereof, to more effectively diminish Type I diabetes symptoms. This treatment should be after the development of at least two autoantibodies indicative of Type I diabetes and before the patient develops high blood sugar. To measure the diminishment of diabetes symptoms, one would typically measure or measure blood sugar. The normal fasting range is 80-120 mg/dl; hyperglycemia (chronic) is reflective of diabetes.

One would most preferably determine whether autoantibodies indicative of Type 1 diabetes are present in a human patient by methods commonly practiced by those of skill in the art: Franke, et al., (Diabetes/Metabolism Research and Reviews, pp. 1-23, 2005, incorporated herein by reference) discloses at Table 1 a set of major autoantibody markers of Type I diabetes. The markers are autoantibodies to islet cells, GAD-65, IA-2, IA-2β and insulin. Autoantibodies to any of these antigens would be suitable for the present invention.

In another embodiment, the method comprises delaying the onset of Type I diabetes patient comprising the step of orally administering and effective amount of vitamin D compound, preferably 1,25(OH)₂D₃ or analogs thereof.

Both methods comprise selecting a Type I diabetes patient or prospective patient and administering a sufficient amount of the vitamin D analog to the patient such that the Type I diabetes symptoms are abated, delayed, or eliminated.

In a particularly advantageous form of the treatment, the administered compound is either 1α,25-dihydroxyvitamin D₃ (1,25-(OH)₂D₃), 19-nor-1,25-dihydroxyvitamin D₂ (19-nor-1,25-(OH)₂D₃), 24-homo-22-dehydro-22E-1α,25-dihydroxyvitamin D₃ (24-homo-22-dehydro-22E-1,25-(OH)₂D₃), 1,25-dihydroxy-24(E)-dehydro-24-homo-vitamin D₃ (1 ,25-(OH)₂-24-homo D₃), 19-nor-1,25-dihydroxy-21-epi-vitamin D₃ (19-nor-1,25-(OH)₂-21-epi-D₃), 1α hydroxy vitamin D₃ or 1α hydroxy vitamin D₂.

In another form of the present invention, the vitamin D compound has the formula R 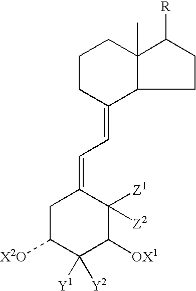
wherein X¹ and X² are each selected from the group consisting of hydrogen and acyl; wherein Y¹ and Y² can be H, or one can be 0-aryl, 0-alkyl, aryl, alkyl of 1-4 carbons, or taken together to form an alkene having the structure of B₁ 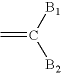
where B₁ and B₂ can be selected from the group consisting of H, alkyl of 1-4 carbons and aryl, and can have a β or α configuration; Z¹=Z²=H or Z¹ and Z² together are ═CH₂; and wherein R is an alkyl, hydroxyalkyl or fluoroalkyl group, or R may represent the following side chain: 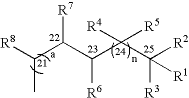
wherein (a) may have an S or R configuration, R¹ represents hydrogen, hydroxy/or 0-acyl/R² and R³ are each selected from the group consisting of alkyl, hydroxyalkyl and fluoralkyl, or, when taken together represent the group —(CH₂)ₘ— wherein m is an integer having a value of from 2 to 5, R⁴ is selected from the group consisting of hydrogen, hydroxy, fluorine, O-acyl, alkyl, hydroxyalkyl and fluoralkyl, wherein if R⁵ is hydroxyl or fluoro, R⁴ must be hydrogen or alkyl, R⁵ is selected from the group consisting of hydrogen, hydroxy, fluorine, alkyl, hydroxyalkyl and fluoroalkyl, or R⁴ and R⁵ taken together represent double-bonded oxygen, R⁶ and R⁷ taken together form a carbon-carbon double bond, R⁸ may be H or CH₃, and wherein n is an integer having a value of from 1 to 5, and wherein the carbon at any one of positions 20, 22, or 23 in the side chain may be replaced by an O, S, or N atom.

We envision that vitamin D compounds of the following formula would be useful:

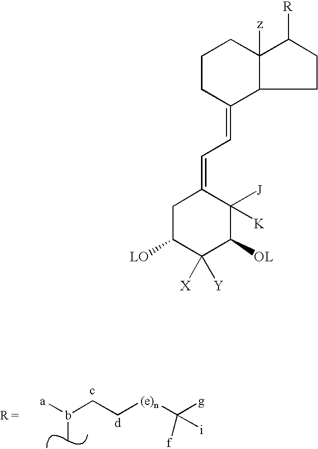

wherein:

J,K=H or methylene

X,Y=H, methylene or alkyl 1-5

z=Ch3, H

L=H or OH protecting group

a,c,d,f,g can be H or alkyl of 1-5

c,d may be H or taken together can be a double bond.

B can be a carbon with S or R configuration

n=integer 1 to 5

I=OH, H.

We envision that a preferable oral dose is as a capsule, tablet, or lozenge that can be included in the diet or may be given in slow release form. Doses of from 0.1 μg to 50 μg/day may be used depending on the particular compound chosen. The dose may also be delivered as a dermal patch, suppository or as a nasal spray and can be given at multiple points or continuously throughout the day.

The present invention is also a pharmaceutical composition comprising an amount of vitamin D compound effective to diminish Type I diabetes symptoms. Preferably, the dose of vitamin D compound is between 0.1 μg and 50 μg/day, most preferably between 0.3 and 12 μg/day. The pharmaceutical composition additionally comprises a pharmaceutically acceptable carrier as is known in the art.

### EXAMPLES

A. Oral Use of 1.25(OH)₂D₃ and Its Analogs in the Prevention of Type I Diabetes in the Non-Obese Diabetic (NOD) Mouse

Materials and Methods

Non-radioactive 1,25(OH)₂D₃ was purchased from Tetrionics, Inc. (Madison, Wis.).

Non-Obese Diabetic (NOD/LtJ) mice were purchased from The Jackson laboratory (Bar Harbor, Me. 04609). Mice were maintained on highly purified vitamin D-deficient diet containing 0.47% calcium and 0.3% phosphorus supplemented with vitamins A, E, and K. This diet was solidified by the addition of molten agar to a powdered diet. To obtain vitamin D-deficiency in the offspring, pregnant mothers were maintained on the vitamin D-deficient diet. Then, offspring, once weaned, were further maintained on the vitamin D-deficient diet. 1,25(OH)₂D₃ was added to the diet at a level such that so each mouse would receive 50 μg/day. Treatment with 1,25(OH)₂D₃ was started at weaning. Animals were bled from the extraorbital sinus with a glass pipette after a four hour fast at 2 pm once a week.

Serum Calcium Determination

Blood was immediately centrifuged to obtain serum. Serum was diluted in 0.1% lanthanum chloride, and serum calcium was measured by atomic absorption using a Perkin-Elmer model 3110 atomic absorption spectrometer.

Serum Glucose Determination

Serum glucose was determined using the Trinder glucose oxidase enzyme assay kit purchased from Sigma (St. Louis, Mo.). 5 μL of NOD serum was used as an unknown and analyzed spectrophotometrically at 505 nm against a known glucose standard (Sigma, St. Louis, Mo.). An animal was considered diabetic if its serum glucose was greater than 300 mg/dL.

Results

FIG. 1 shows the incidence of diabetes in the vitamin D-deficient NOD mouse colony compared to the NOD mice treated with 1,25(OH)₂D₃. At the termination of this experiment, the incidence of diabetes in the male vitamin D-deficient NOD mice was 80%. By contrast, when the mice were treated with 50 ng/mouse/day of 1,25(OH)₂D₃, the incidence of diabetes in the female NOD mice dropped drastically to 7.69% and the incidence in the male NOD mice dropped to 7.14%. Therefore, treatment with 1 ,25(OH)₂D₃ could largely prevent autoimmune diabetes in the NOD mouse.

FIG. 2 depicts the serum calcium data from these mice over the time course of this experiment. The vitamin D-deficient mouse were hypocalcemic at the beginning of the experiment (males=6.69±0.9 mg/dL and females=6.38±1.38 mg/dL). Their serum calcium gradually increased during the experiment to reach a value of approximately 8 mg/dL in both males and females by the end of the experiment. In the mice treated with 50 ng 1,25(OH)₂D₃ each day, serum calcium values were significantly higher. At 40 days, the males administered this treatment had a serum calcium of 9.22±0.93 mg/dL while the females had a serum calcium of 10.50±1.53 mg/dL. This level of serum calcium also increased over the time of the experiment. At 150 days of age, males had serum calcium values of 12.78±1.11 mg/dL while females had serum calcium values of 13.10±1.01 mg/dL. The females were smaller than the males in this experiment; therefore, they were affected by this hypercalcemia to a greater extent. By 150 days of age, 7 of the NOD females treated with 50 ng 1,25(OH)₂D₃ had died of hypercalcemia. It was decided at this time to reduce the 1,25(OH)₂D₃ amount in the diet to 10 ng/day in both the males and females. Doses of 200 ng 1,25(OH)₂D₃ were originally administered to another group of NOD mice, but females began dying of hypercalcemia at 100 days of age and males began dying of hypercalcemia at 120 days of age. Evidence of hypercalcemia was extremely high serum calcium levels (≧12 mg/dL) and kidney calcification indicated by white spots in the kidney. Also, at no time did any of these animals present serum glucose values greater than 300 mg/dL.

Discussion

The incidence of diabetes in vitamin D-deficient NOD mice was compared to that in NOD mice treated with 1,25(OH)₂D₃. As shown in FIG. 1, 50 ng of 1,25(OH)₂D₃ administered in the diet could prevent diabetes in both male and female NOD mice. This prevention of diabetes occurred at the expense of hypercalcemia, as shown in FIG. 2. These data are evidence of the therapeutic potential of 1,25(OH)₂D₃ in the prevention of Type I diabetes. Of course, an analog(s) of 1,25(OH)₂D₃ will preferably be used that will be immunoreactive without causing high serum calcium levels.

One remarkable observation from the data in FIG. 1 is that the incidence in the vitamin D-deficient population of NOD mice was significantly higher than that reported in the literature for chow-fed animals. Previously published data indicated that 70-80% of chow-fed female NOD mice and 20% of chow-fed male NOD mice developed diabetes (S. Makino, et al., supra, 1981). Here, the incidence of disease in females was 91.67% while the incidence in males was 80%. Therefore, the absence of vitamin D from an otherwise nutritionally complete diet potentiated the development of diabetes. This indicates that at least one of the environmental factors contributing to the development of diabetes in the NOD mouse is a lack of vitamin D.

Therapeutically, we propose that this treatment can be used in children predisposed to the development of diabetes. These children would be those with autoantibodies to β cell antigens. There are two well-known β cell antigens including: glutamic acid decarboxylase and insulin (S. Baekkeskov, et al., supra, 1990; W. A. Hagopian, et al., supra, 1993; L. Castano and G. S. Eisenbarth, supra, 1990). If autoantibodies can be detected in prediabetic children, treatment with 1,25(OH)₂D₃ can be started early, and diabetes can be prevented.

B. Injection vs. Dietary Study

We compared injectable vs. oral treatment of 1,25(OH)₂D₃ to determine whether dietary administration or i.p. injection of 1,25(OH)₂D₃ is more effective in preventing diabetic onset in female NOD/LtJ mice.

NOD/LtJ mice were fed control diets (purified Diet 11 as described in Suda, et al., J. Nutr. 100:1049-1052 1970, 0.47% calcium +vitamins A, D, E and K) or experimental diets (control +10, 25 or 50 ng of 1,25D₃/mouse/day).

Control (vehicle) animals were injected with 50 μL sterile peanut oil while the experimental animals were injected with 5 μg/kg 1,25D₃/mouse/2 days in sterile peanut oil.

Mice were weaned at 21 days and placed on the appropriate diets. Injections and experimental diets were administered every 48 hours. Control diets were fed 3 times/week. Every 10 days beginning at day 30 and ending on day 170, mice were weighed and bled for serum calcium determination. Beginning on day 70, mice were tested for glucosuria 3 times/week. If a mouse tested positive, it was fasted for 4 hours and bled to measure serum glucose levels. If the fasting serum glucose level was greater than 300 mg%, the mouse was considered diabetic. All mice remaining at 200 days were sacrificed.

Results

Table 1 and FIG. 3, a bar graph describing diabetic incidence at day 200 in the female NOD/LtJ mice describe the results. The mice with injected 1,25(OH)₂D₃ had an over 70% incidence of diabetes. Vehicle injected and mice with no added vitamins D compound had over 40% and 50% incidence of diabetes. In contrast, the mice with 1,25(OH)₂D₃ in the diet (oral treatment) had a diabetes incidence of between 10-25%. The lower dose of 1,25(OH)₂D₃ is likely closer to the optimal dose for preventing diabetes. The high doses clearly caused hypercalcemia but nevertheless reduced the incidence of diabetes. More important, in our hands injection of 50 μg/day increased rather than decreased the incidence of diabetes.

It is possible that lower oral dose of 1 ,25(OH)₂D₃ will be effective without causing hypercalcemia. The data are nevertheless sufficient to conclude that oral and not injected dosages of 1-hydroxylated vitamin D will prevent the onset of type 1 diabetes.

TABLE 1
Injection vs. Dietary Study - Day 200 Results

| Group | n= | db n= | db %= |
|---|---|---|---|
| 1.25 inject | 7 | 5 | 71 |
| vehicle inject | 7 | 3 | 43 |
| 0 ng diet | 9 | 5 | 56 |
| 10 ng diet | 9 | 1 | 11 |
| 25 ng diet | 9 | 2 | 22 |
| 50 ng diet | 8 | 2 | 25 |

FIG. 4 graphs the day of onset of diabetes in the female NOD/LtJ mice.

C. Treatment of Diabetes After the Appearance of Autoantibodies to Pancreatic Products

In non-obese diabetic (NOD) mice, autoantibodies to pancreatic products such as insulin peak in the serum beginning at 8 weeks of age. Presence of these antibodies at 8 weeks of age is strongly associated with future development of diabetes.

In humans, autoantibodies to insulin (GAD-65) and insulinoma-associated protein 2 (IA-2) are normally present in the serum before overt diabetes. These autoantibodies have been monitored in offspring of parents with autoimmune diabetes. Cumulative risk of diabetes is less than 2% in these patients, while the presence of 2 or more autoantibodies by age 2 in these patients increases the risk of developing diabetes to 50% by age 5.

In humans these autoantibodies have a strong predictive value for future diabetes development. A preventative compound would be ideal if it could be administered to these patients after these autoantibodies appear in the serum but before overt diabetes develops. Here we show that 50 ng 1,25(OH)₂D₃ protects NOD mice from autoimmune diabetes when administered beginning at 8 weeks of age when autoantibodies can be detected in the serum. Accordingly, 1,25(OH)₂D₃ or analogs of 1,25(OH)₂D₃ could be used to prevent autoimmune diabetes in human patients positive for these autoantibodies.

Materials and Methods

Animals: Mice from an in-house breeding colony of NOD/LtJ mice (Jackson Laboratory) were used for this study. Female mice were randomly weaned into each dietary group. 24 mice were used per group.

Diet Preparation: Experimental purified diets were prepared containing 0.47% calcium supplemented with 25 IU vitamin D3 (Diet 11). 1,25(OH)₂D₃ was added to 1,25(OH)₂D₃-containing diet through the fat source (vegetable oil). 1,25(OH)₂D₃ was added such that 3.5 g powder diet contained 50 ng of 1,25(OH)₂D₃. All purified powder diets were solidified by mixing with molten agar to ensure animals consumed the exact dose of 1,25(OH)₂D₃. Each mouse was administered 3.5 grams of powder diet per day.

Diet Administration: Experimental purified diets were administered beginning at weaning (3 weeks of age) until 200 days of age. 1,25(OH)₂D₃-containing diet was administered beginning at weaning (group 2), beginning at 8 weeks of age (group 3), or beginning at 100 days of age (group 4).

| | Diet | | |
|---|---|---|---|
| Group | Weaning-8 weeks | 8 weeks-100 days | 100 days-200 days |
| 1 | Diet 11 | Diet 11 | Diet 11 |
| 2 | Diet 11 + 50 ng | Diet 11 + 50 ng | Diet 11 + 50 ng |
| | 1.25(OH)₂D₃ | 1.25(OH)₂D₃ | 1.25(OH)₂D₃ |
| 3 | Diet 11 | Diet 11 + 50 ng | Diet 11 + 50 ng |
| | | 1.25(OH)₂D₃ | 1.25(OH)₂D₃ |
| 4 | Diet 11 | Diet 11 | Diet 11 + 50 ng |
| | | | 1.25(OH)₂D₃ |

Serum calcium determination: Serum calcium levels were measured at various time points to ensure correct delivery of vitamin D compounds. Serum samples were diluted 1:40 in 0.1% LaCl₃ and calcium levels were measured by atomic absorption spectrometry.

Serum glucose determination: Serum glucose was measured using an enzymatic colorimetric assay (Glucose liquicolor, Stanbio, Boerne, Tex.).

Disease analysis: Urine samples were obtained twice weekly from animals older than 70 days of age and tested for urinary glucose using Diastix (Bayer, Elkhart, Ind.). Serum glucose was measured from animals with urine glucose readings >250 mg/dL. Animals with multiple serum glucose values >300 mg/dL at least 24 hours apart were considered diabetic and sacrificed via CO₂ asphyxiation. All remaining animals were sacrificed at 200 days of age.

Results

Diabetes Incidence: Seven of twenty-four (29.2%) untreated mice became diabetic by 200 days of age. One of twenty four (4.2%) of mice treated with 50 ng 1,25(OH)₂D₃ became diabetic by 200 days of age. No animals (0 of 24) receiving 50 ng 1,25(OH)₂D₃ beginning at 8 weeks of age became diabetic by 200 days of age. Two of twenty-three (8.7%) of animals receiving 50 ng 1,25(OH)₂D₃ after 100 days of age became diabetic by 200 days of age. FIG. 5 graphs these results.

Diabetes incidence in group 2 (50 ng 1,25(OH)₂D₃ from weaning) and group 3 (50 ng 1,25(OH)₂D₃ beginning at 8 weeks of age) were significantly different from group 1 (untreated), P<0.05 for group 2, P<0.01 for group 3 by Fisher's exact test.

Serum Calcium: Serum calcium levels were raised by roughly 2.0 to 2.4 mg/dL by the administration of 50 ng 1 ,25(OH)₂D₃. The calcemic effect of 50 ng 1,25(OH)₂D₃ was evident within 1 week of administration. FIG. 6 graphs these results.

Weight: The addition of 50 ng 1,25(OH)₂D₃ to group 2 animals decreased body weight after 8 weeks. The addition of 50 ng 1 ,25(OH)₂D₃ after either 8 weeks or 100 days of age caused a decrease in body weight compared to untreated animals, but the effect was not as severe as when 50 ng 1 ,25(OH)₂D₃ was administered from weaning until 200 days of age. FIG. 5 is a bar graph describing diabetes incidence among four groups of NOD rats. The groups differ in the amount of 1,25(OH)₂D₃ that has been administered and the time of administration. FIG. 6 is a bar graph showing serum calcium in the four groups of FIG. 5. FIG. 7 graphs body weight as a function of age in the four groups of FIG. 5.

### SUMMARY

These results show that 1,25(OH)₂D₃ prevents diabetes in NOD mice when administered at weaning through 200 days of age or from 8 weeks of age until 200 days of age. Administration of 1,25(OH)₂D₃ after 100 days of age reduced the incidence of diabetes, but the difference did not reach statistical significance.

We claim:

1. A method of delaying the onset of diabetes in a human patient, comprising the step of orally administering to the patient an effective amount of a vitamin D compound such that the onset of Type I diabetes or diabetes symptoms is slowed, wherein the treatment is after the appearance and detection of at least two autoantibodies indicative of Type I diabetes and before the patient develops blood sugar of 150 mg/dL, measured as an average of 5 samples taken throughout the day at least 2 hours apart.

2. The method of claim 1 wherein the autoantibodies are specific for an antigen selected for the group consisting of islet cells, GAD-65, IA-2, IA-2β and insulin.

3. The method of claim 1 wherein the compound is selected from the group consisting of 1α,25-dihydroxyvitamin D₃ (1,25-(OH)₂D₃), 19-nor-1,25-dihydroxyvitamin D₂ (19-nor-1,25-(OH)₂D₃), 24-homo-22-dehydro-22E-1α,25-dihydroxyvitamin D₃ (24-homo-22-dehydro-22E-1,25-(OH)₂D₃), 1,25-dihydroxy-24(E)-dehydro-24-homo-vitamin D₃ (1,25-(OH)2-24-homo D₃), 19-nor-1,25-dihydroxy-21 -epi-vitamin D₃ (19-nor-1,25-(OH)₂-21 -epi-D₃),1α hydroxy vitamin D₃ or 1α hydroxy vitamin D₂.

4. The method of claim 1 wherein the vitamin D compound is selected from the group consisting of vitamin D compounds with the following formula: 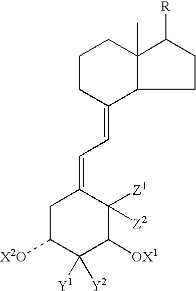
wherein X¹ and X² are each selected from the group consisting of hydrogen and acyl;
wherein Y¹ and Y² can be H, or one can be 0-aryl, 0-alkyl, aryl, alkyl of 1-4 carbons, taken together to form an alkene having the structure of B₁ 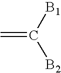
where B₁ and B₂ can be selected from the group consisting of H, alkyl of 1-4 carbons and aryl, and can have a β or α configuration; Z¹=Z²=H or Z¹ and Z² together are ═CH₂; and wherein R is an alkyl, hydroxyalkyl or fluoroalkyl group, or R may represent the following side chain: 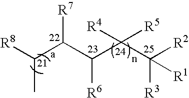
wherein (a) may have an S or R configuration, R¹ represents hydrogen, hydroxy or O-acyl, R² and R³ are each selected from the group consisting of alkyl, hydroxyalkyl and fluoralkyl, or, when taken together represent the group —(CH₂)ₘ— wherein m is an integer having a value of from 2 to 5, R⁴ is selected from the group consisting of hydrogen, hydroxy, fluorine, O-acyl, alkyl, hydroxyalkyl and fluoralkyl, wherein if R⁵ is hydroxyl or fluoro, R⁴ must be hydrogen or alkyl, R⁵ is selected from the group consisting of hydrogen, hydroxy, fluorine, alkyl, hydroxyalkyl and fluoroalkyl, or R⁴ and R⁵ taken together represent double-bonded oxygen, R⁶ and R⁷ taken together form a carbon-carbon double bond, R⁸ may be H or CH₃, and wherein n is an integer having a value of from 1 to 5, and wherein the carbon at any one of positions 20, 22, or 23 in the side chain may be replaced by an 0, S, or N atom.

5. The method of claim 1 wherein the oral administration is via diet.

6. The method of claim 1 wherein the oral administration is at the concentration of between 0.005 μg to 0.2 μg per kilogram of patient weight per day.

7. A method of reducing the severity of diabetes symptoms comprising orally administering to a human diabetes patient an effective amount of vitamin D compounds such that diabetes symptoms are lessened.

8. The method of claim 6 wherein the compound is selected from the group consisting of 1α,25-dihydroxyvitamin D₃ (1,25-(OH)₂D₃), 19-nor-1,25-dihydroxyvitamin D₂ (19-nor-1,25-(OH)₂D₃), 24-homo-22-dehydro-22E-1α,25-dihydroxyvitamin D₃ (24-homo-22-dehydro-22E-1,25-(OH)₂D₃),1,25-dihydroxy-24(E)-dehydro-24-homo-vitamin D₃ (1,25-(OH)₂-24-homo D₃), 19-nor-1,25-dihydroxy-21-epi-vitamin D₃ (19-nor-1,25-(OH)₂-21-epi-D₃), 1α hydroxy vitamin D₃ or 1α hydroxy vitamin D₂.

9. The method of claim 6 wherein the vitamin D compound is selected from the group consisting of vitamin D compounds with the following formula: 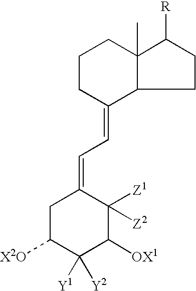
wherein X¹ and X² are each selected from the group consisting of hydrogen and acyl;
wherein Y¹ and Y² can be H, or one can be 0-aryl, 0-alkyl, aryl, alkyl of 1-4 carbons, taken together to form an alkene having the structure of B₁ where B₁ and B₂ can be selected from the group consisting of H, 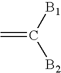
alkyl of 1-4 carbons and aryl, and can have a β or α configuration; Z¹=Z²=H or Z¹ and Z² together are =CH₂; and wherein R is an alkyl, hydroxyalkyl or fluoroalkyl group, or R may represent the following side chain: 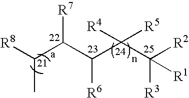
wherein (a) may have an S or R configuration, R¹ represents hydrogen, hydroxy or O-acyl, R² and R³ are each selected from the group consisting of alkyl, hydroxyalkyl and fluoralkyl, or, when taken together represent the group —(CH₂)ₘ— wherein m is an integer having a value of from 2 to 5, R⁴ is selected from the group consisting of hydrogen, hydroxy, fluorine, 0-acyl, alkyl, hydroxyalkyl and fluoralkyl, wherein if R⁵ is hydroxyl or fluoro, R⁴ must be hydrogen or alkyl, R⁵ is selected from the group consisting of hydrogen, hydroxy, fluorine, alkyl, hydroxyalkyl and fluoroalkyl, or R⁴ and R⁵ taken together represent double-bonded oxygen, R⁶ and R⁷ taken together form a carbon-carbon double bond, R⁸ may be H or CH₃, and wherein n is an integer having a value of from 1 to 5, and wherein the carbon at any one of positions 20, 22, or 23 in the side chain may be replaced by an O, S, or N atom.

10. The method of claim 6 wherein the oral administration is via diet.

11. The method of claim 6 wherein the oral administration is at the concentration of between 0.005 μg to 0.2 μg per kilogram of patient weight per day.

12. A method of claim 1 wherein the vitamin D compound is selected from the group consisting of 1α,25-dihydroxyvitamin D₃, 1α hydroxyvitamin D₃, 1α,25-dihydroxyvitamin D₂, 1α-hydroxyvitamin D₂, 19-nor-1α-hydroxyvitamin D₂, 22-oxa-1α,25 dihydroxyvitamin D₃, 26,27-hexaflouro-1α,25 dihydroxyvitamin D₃, 24R-hydroxy-26,27-cyclo-1α hydroxyvitamin D₃, 2-methylene-19-nor-(20S)-1α,25 dihydroxyvitamin D₃, 2 methylene-19 nor-20S-1α,25 cihydroxy vitamin D₃ (2MD), 2α methyl-19 nor-20S-1α,25 hydroxyvitamin D₃, and their 20R isomers, 2 methylene-19-1α-hydroxyl-homopregnacalciferol, 2 methylene-19-nor-1α-hydroxy-(20S,20R)-bishomopregnacalciferol, and 2-methylene-19-nor-1α-hydroxypregnacalciferol.

13. The method of claim 1 wherein the vitamin D compound is characterized by the following formula: 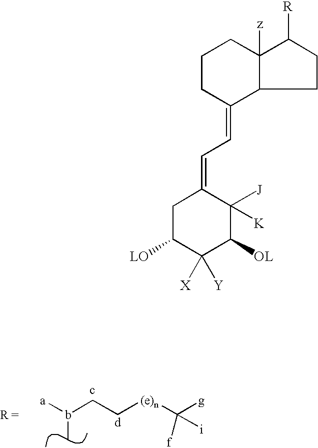
wherein:
J,K=H or methylene
X,Y=H, methylene or alkyl 1-5
z=Ch3, H
L=H or OH protecting group
a,c,d,f,g can be H or alkyl of 1-5
c,d may be H or taken together can be a double bond.
B can be a carbon with S or R configuration
n=integer 1 to 5
I=OH, H.
